(19) United States
(12) Patent Application Publication
(10) Pub. No.: US 2014/0199372 A1
(43) Pub. Date: Jul. 17, 2014

(54) MICROBIAL GROWTH FACTORS

(71) Applicant: NORTHEASTERN UNIVERSITY, Boston, MA (US)

(72) Inventors: Kim Lewis, Newton, MA (US); Pallavi Murugkar, Boston, MA (US); Anthony D'onofrio, Northborough, MA (US); Eric Stewart, Swampscott, MA (US); Eric Dimise, Brighton, MA (US); Jon Clardy, Jamaica Plain, MA (US); Kathrin Witt, Cambridge, MA (US); Bijaya Sharma, Revere, MA (US)

(73) Assignee: NORTHEASTERN UNIVERSITY, Boston, MA (US)

(21) Appl. No.: 14/119,566

(22) PCT Filed: May 24, 2012
(86) PCT No.: PCT/US12/39335

§ 371 Date: Mar. 6, 2014

Related U.S. Application Data
(60) Provisional application No. 61/489,371, filed on May 24, 2011.

(51) Int. Cl. C12N 1/38; A61K 9/127; A61K 35/74; A61K 31/122

(52) U.S. Cl. CPC C12N 1/38; A61K 31/122; A61K 9/127; A61K 35/741
USPC 424/450; 435/244; 514/678; 424/93.4; 514/682

(57) ABSTRACT

Disclosed are methods of cultivating or isolating a microorganism using one or more quinones as growth factors. Also disclosed are methods of treating a mammalian species with deficiency in symbionts using such compounds.

This application claims priority to U.S. Provisional Patent Application No. 61/489,371, filed May 24, 2011, the contents of which are hereby incorporated by reference in their entireties.

### BACKGROUND

The GenBank® sequence database, which is an annotated collection of all publicly-available nucleotide and amino acid sequences, contains sequences from approximately 30,000 species of bacteria. While this number may appear impressive, it is instructive to note that a recent estimate suggests that the sea may support as many as 2 million different species of bacteria, and a ton of soil more than double that number (Curtis et al., Proc. Natl. Acad. Sci. USA 99:10494-10499, 2002). Furthermore, only about 13,000 of the bacteria represented in GenBank® have been formally described, and almost all of these lie within 4 of the 40 bacterial divisions (DeLong, Curr. Opin. Microbiol. 4:290-295, 2001). The paucity of knowledge regarding other microbial species is similar or greater. This is at least in part due to the fact that the vast majority of microorganisms from the environment resist cultivation in the laboratory. These so called “uncultivables” represent 99-99.99% of all microbial species in nature (see, e.g., Young, ASM News 63:417-421, 1997).

Microbial diversity is typically examined by amplifying 16S rRNA genes from DNA samples isolated from a specific habitat. The sequences are then compared to each other and to the 16S rRNA sequences from known species. If no close match to an existing 16S rRNA gene sequence is found, then the test sequence is thought to represent a new microorganism and is termed an “uncultured microorganism.” 16S rRNA genes, which are critical for translation, are the genes of choice for these experiments because they are thought to be conserved across vast taxonomic distance, yet show some sequence variation between closely related species. Phylogenetic analyses of 16S rRNA sequences obtained from direct sampling of environments suggest that uncultured microorganisms can be found in nearly every taxon within Bacteria and Archaea, and several groups at the division level have been identified with no known cultivable representatives (see, e.g., Giovannoni et al., Nature 345: 60-63, 1990; and Dojka et al., Appl. Environ. Microbiol. 66:1617-1621, 2000).

The principal reason for this disparity is that few microorganisms from environmental samples grow on nutrient media in Petri dishes. The discrepancy between the microbial total count and plate count is several orders of magnitude. Attempts to improve the recovery of microorganisms from environmental samples by manipulating growth media have been of limited success.

Researchers have used a variety of media in hopes of growing previously uncultivated microorganisms but haven't been able to grow all the organisms from a given environment. Menadione, a synthetic quinone, has been added to media used to grow organisms from the human microbiome but it hasn't been able to grow a significant number of organisms from this environment.

Accordingly, new methods for isolating and growing previously uncultivable microorganisms are desirable. These methods may be useful in identifying microorganisms that are a valuable resource of novel metabolic products useful for pharmaceutical and industrial processes. In addition, these methods may be useful in identifying microorganisms critical for decomposing and recycling nutrients at a global scale.

### SUMMARY OF THE INVENTION

The present disclosure is directed to the use of quinones as growth factors for previously uncultured microorganisms. The majority of environmental bacteria are uncultured, do not grow in the laboratory on standard growth media, and a considerable part of microorganisms inhabiting humans (the Microbiome) are uncultured as well. Environmental microorganisms are a potential source of valuable secondary metabolites, and uncultured microorganisms from the human microbiome are potential symbionts. Finding growth factors for uncultured microorganisms is therefore of considerable utility. Specifically, the quinone growth factors described in this invention may be used to treat humans with deficiency in certain symbionts. Quinones were found to be essential for growth of uncultured bacteria, but may also be useful to stimulate the growth of desirable cultivable species. Quinones may also be added to growth media for growing uncultured environmental microorganisms for the production of secondary metabolites such as antibiotics.

This is a novel approach as organisms which require an exogenous quinone-type compound have been previously thought to be able to utilize menadione, however the recommended media containing menadione are not capable of growing the organism described here, and this is likely to be the case for other significant bacteria.

In one aspect, the present disclosure is directed to a method for cultivating or isolating a microorganism, the method comprising using one or more quinones as growth factors.

In another aspect, the present disclosure is directed to a method for treating a mammalian species with deficiency in symbionts, the method comprising administering to the mammalian species a therapeutically effective amount of one or more quinones.

These and other embodiments of the invention are further described in the following sections of the application, including the Detailed Description, Examples, and Claims. Still other objects and advantages of the invention will become apparent by those of skill in the art from the disclosure herein, which are simply illustrative and not restrictive. Thus, other embodiments will be recognized by the ordinarily skilled artisan without departing from the spirit and scope of the invention.

### BRIEF DESCRIPTION OF THE FIGURES

FIG. 1 shows growth induction of KLE1280. (A) KLE1280 growing near a mix of helpers from the oral microbiome; (B) KLE1280 growing around a spot of WT Escherichia coli; (C) E. coli mutant OCL67 not inducing growth of KLE1280.

FIG. 2 shows genes knocked out in E. coli mutant OCL67. The shaded region depicts the knockout mutant OCL67. 5 genes in the menaquinone (MK) biosynthesis pathway are knocked out in this mutant.

FIG. 3 shows the menaquinone biosynthetic pathway of Porphyromonas gingivalis W83.

FIG. 4 shows induction of growth of KLE1215 by E. coli. KLE1215 spread evenly on R2A with 50% artificial sea salts and 0.001% Fe(II) with a dense spot of E. coli.

FIG. 5 shows E. Coli ΔubiG, a strain deficient in quinone biosynthesis, does not induce the growth of KLE1215. KLE1215 spread evenly on R2A with 50% artificial sea salts and 0.001% Fe(II) with a dense spot of E. coli ΔubiG.

FIG. 6 shows Shewanella oneidensis MR-1 ZK2719 induces growth of KLE1215. KLE1215 spread evenly on R2A with 50% artificial sea salts and 0.001% Fe(II) with a dense spot of Shewanella MR-1 ZK2719.

FIG. 7 shows Shewanella sp. ΔmenC:kan ZK2720, deficient in quinone production, does not induce growth of KLE1215. KLE1215 spread evenly on R2A with 50% artificial sea salts and 0.001% Fe(II) with a dense spot of Shewanella sp. ΔmenC:kan ZK2720.

FIG. 8 shows growth of KLE1215 induced with varying concentrations of MK4 delivered in liposomes.

FIG. 9 shows growth of KLE1215 induced by MK6 and MK6 chromenol purified from KLE1215 delivered in liposomes.

FIG. 10 shows aerobic helper-dependent pair isolated from marine sand biofilm. Bizinio sp. KLE1402, on the right, is induced to grow by a helper (Ruegeria sp. KLE1403) from the same environment.

FIG. 11 shows E. coli induces the growth of KLE1402. KLE1402 spread evenly on R2A with 50% artificial sea salts and 0.001% Fe(II) with a dense spot of E. coli.

FIG. 12 shows E. coli ΔubiG, a strain deficient in quinone biosynthesis, does not induce the growth of KLE1402. KLE1402 spread evenly on R2A with 50% artificial sea salts and 0.001% Fe(II) with a dense spot of E. coli ΔubiG.

FIG. 13 shows KLE1402 growth with the addition of quinone-loaded liposomes. Cells of KLE1402 were used to inoculate culture tubes with 5 mL R2A broth with 50% sea salts and 0.001% Fe(II). Tube A: Nothing added, Tube B: Empty liposomes added, Tube C: Liposomes loaded with 500 μM MK4 added.

FIG. 14 shows KLE1402 growth with the addition of quinone-loaded liposomes. Cultures of KLE1402 with no supplement (squares), empty liposomes as liposome control (triangles), and liposomes loaded with 500 μM MK4 (cross).

FIG. 15 shows isolation of the helper-dependent gut isolate Faecalibacterium sp. KLE1255. (A) The original isolation plate showing colonies from a fecal sample growing on BHIych with a spot of E. coli. Colonies growing in close proximity to the E. coli spot were picked, and tested for dependency on the helper, by (B) spreading the potential dependent isolate evenly onto the plate and spotting E. coli, and by (C) streaking the two organism close to each other.

FIG. 16 shows E. coli deletion mutant OCL67 fails to induce growth of Faecalibacterium sp. KLE1255. (A) The image shows the chromosomal region deleted in E. coli strain OCL67 (16.4 kb, shaded in red) (PEC database: www.shigen.nig.ac.jp/ecoli/pec/index.jsp). The deletion includes 16 genes, six of which (menBCDEFH) make up the menaquinone biosynthesis operon. (B) A tight ring of growth of KLE1255 can be observed around wild-type E. coli. (C) No growth of KLE1255 can be observed with the E. coli mutant OCL67.

FIG. 17 shows menaquinone-like compounds from Micrococcus luteus supernatant act as growth factors. Four fractions from M. luteus supernatant hexane extract induced the growth of KLE1255. Two of the fractions contain menaquinone 4 and preliminary data suggest that a third fraction (Mlu-hex-6) also contains a menaquinone-like compound. (A) Structure of menaquinone. The side chain consists of isoprenoid residues and can vary in length. n specifies the number of isoprenoid repeats. (B) Growth induction of KLE1255 by the fraction Mlu-hex-6, containing a menaquinone-like compound.

### DETAILED DESCRIPTION

In one aspect, the present disclosure is directed to a method for cultivating or isolating a microorganism, the method comprising using one or more quinones as growth factors.

In another aspect, the present disclosure is directed to a method for treating a mammalian species with deficiency in symbionts, the method comprising administering to the mammalian species a therapeutically effective amount of one or more quinones.

In some embodiments, the methods further comprise a helper strain microorganism. In some embodiments, the quinone is produced by the helper strain microorganism. In some embodiments, the quinones are delivered in liposomes.

In some embodiments, the quinones are selected from MK4, MKS, MK6, and 1,4-dihydroxy-2-naphthoate (DHNA).

In some embodiments, the cultivated or isolated microorganism is selected from Flaviramulus sp., Bizinio sp., Porphyromonas sp., and Faecalibacterium sp. In some embodiments, the cultivated or isolated microorganism is from Flaviramulus sp. In some embodiments, the cultivated or isolated microorganism is from Bizinio sp. In some embodiments, the cultivated or isolated microorganism is from Porphyromonas sp. In some embodiments, the cultivated or isolated microorganism is from Faecalibacterium sp.

In some embodiments, the cultivated or isolated microorganism is selected from Flaviramulus sp. KLE1215, Bizinio sp. KLE1402, Bizionia echini, Porphyromonas sp. KLE1280, Porphyromonas catoniae, Porphyromonas gingivalis, Faecalibacterium prausnitzii and Faecalibacterium sp. KLE1255. In some embodiments, the cultivated or isolated microorganism is Flaviramulus sp. KLE1215. In some embodiments, the cultivated or isolated microorganism is Bizinio sp. KLE1402. In some embodiments, the cultivated or isolated microorganism is Bizionia echini. In some embodiments, the cultivated or isolated microorganism is Porphyromonas sp. KLE1280. In some embodiments, the cultivated or isolated microorganism is Porphyromonas catoniae. In some embodiments, the cultivated or isolated microorganism is Porphyromonas gingivalis. In some embodiments, the cultivated or isolated microorganism is Faecalibacterium prausnitzii. In some embodiments, the cultivated or isolated microorganism is Faecalibacterium sp. KLE1255.

In some embodiments, the symbiont is selected from Flaviramulus sp., Bizinio sp., Porphyromonas sp., and Faecalibacterium sp. In some embodiments, the symbiont is from Flaviramulus sp. In some embodiments, the symbiont is from Bizinio sp. In some embodiments, the symbiont is from Porphyromonas sp. In some embodiments, the symbiont is from Faecalibacterium sp.

In some embodiments, the symbiont is selected from Flaviramulus sp. KLE1215, Bizinio sp. KLE1402, Bizionia echini, Porphyromonas sp. KLE1280, Porphyromonas catoniae, Porphyromonas gingivalis, Faecalibacterium prausnitzii and Faecalibacterium sp. KLE1255. In some embodiments, the symbiont is Flaviramulus sp. KLE1215. In some embodiments, the symbiont is Bizinio sp. KLE1402. In some embodiments, the symbiont is Bizionia echini. In some embodiments, the symbiont is Porphyromonas sp. KLE1280. In some embodiments, the symbiont is Porphyromonas catoniae. In some embodiments, the symbiont is Porphyromonas gingivalis. In some embodiments, the symbiont is Faecalibacterium prausnitzii. In some embodiments, the symbiont is Faecalibacterium sp. KLE1255.

In some embodiments, the helper strain microorganism is selected from Escherichia coli, Shewanella oneidensis, Ruegeria lacuscaerulensis, and Micrococcus luteus. In some embodiments, the helper strain microorganism is selected from Escherichia coli, Shewanella oneidensis, and Micrococcus luteus. In some embodiments, the helper strain microorganism is selected from Escherichia coli and Shewanella oneidensis. In some embodiments, the helper strain microorganism is Escherichia coli. In some embodiments, the helper strain microorganism is Shewanella oneidensis. In some embodiments, the helper strain microorganism is Ruegeria lacuscaerulensis. In some embodiments, the helper strain microorganism is Micrococcus luteus.

It will be recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Induction of Growth of KLE1280

Isolation of uncultured bacteria of the oral Microbiome: the principal method of isolation was co-culture, placing cultivable bacteria that may produce growth factors on a plate containing uncultured species, and observing organisms that only grow in the presence of helper species.

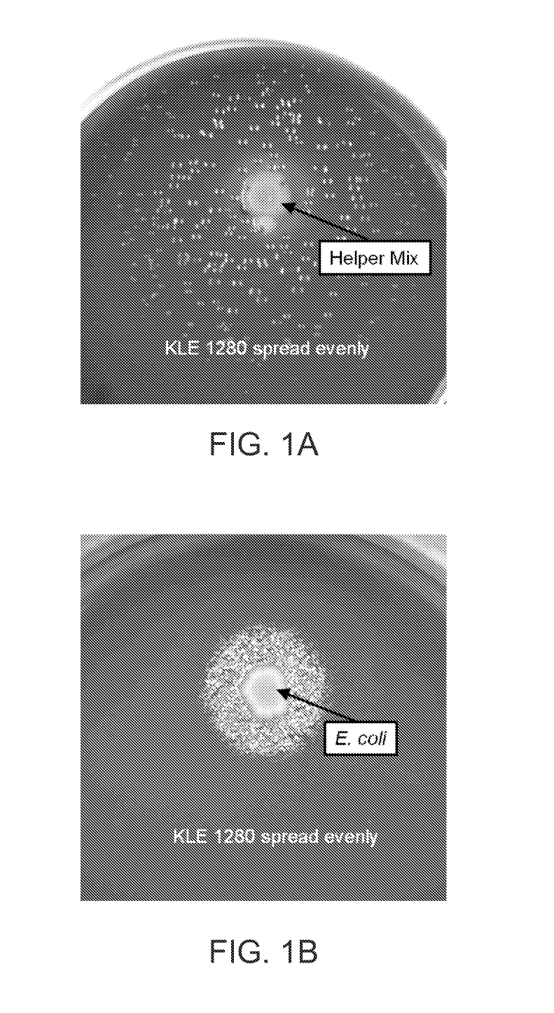

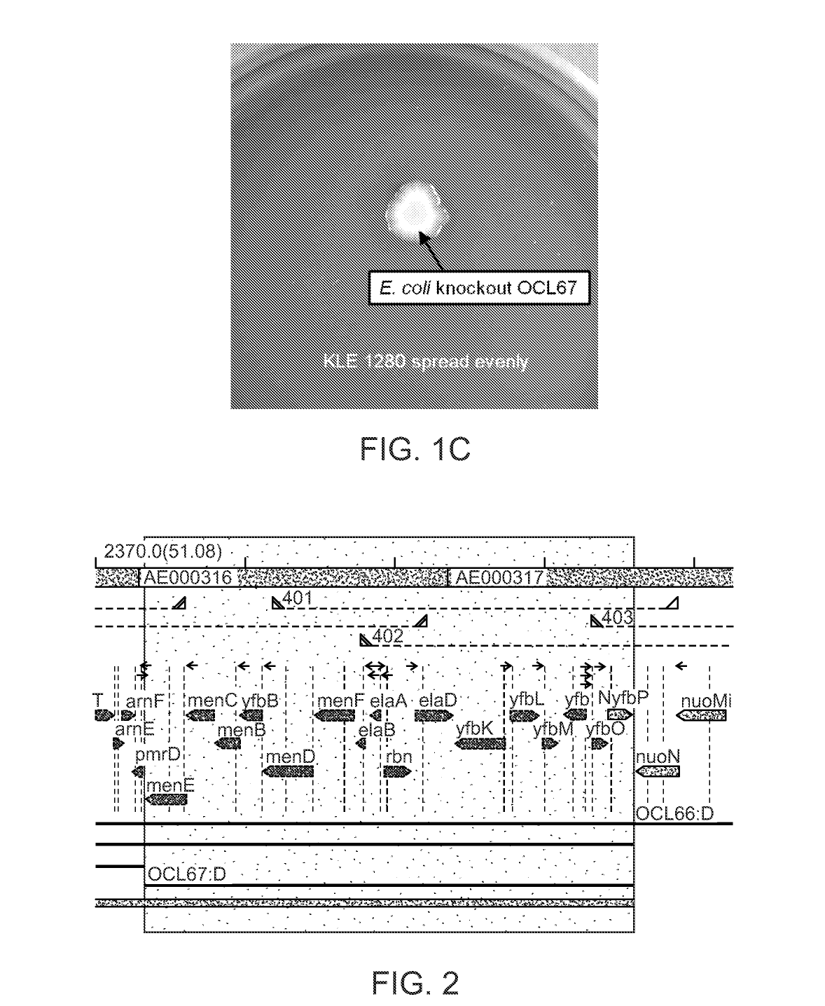

Serial dilutions of dental plaque from a healthy individual were spread onto Fastidious Anaerobe Agar plates containing 5% sheep blood and 5% pooled human saliva (FBS) and incubated anaerobically. Small colonies growing next to large ones on dense plates were picked and spread onto fresh FBS plates. A helper mix of colonies growing around individual small colonies was spotted on these plates. Isolates were identified that depended for growth on this mix. If E. coli produces a growth factor, its identification can be efficiently performed, since a complete knockout library of this species is available. Testing knockout mutants identifies those that lost their ability to help growth of the uncultured organism, which then leads to the identification of the biosynthetic pathway of the growth factor. One of the isolates, KLE1280, showed dependence on the mix or E. coli. This organism is closely related to Porphyromonas sp. oral taxon 279 (99% similarity by 16S rRNA gene sequencing) (96% similarity to closest type strain Porphyromonas catoniae). In particular, KLE1280 is closely related to Porphyromonas catoniae (96% similarity by 16S rRNA gene sequencing). Medium and large deletion mutants of nonessential genes of E. coli were tested as helpers to determine which knockout did not induce growth of KLE1280. E. coli strain OCL67 showed no induction (FIG. 1). This deletion mutant has the menaquinone biosynthesis genes deleted. FIG. 2 shows that the OCL67 deletion removes the menaquinone biosynthesis operon. 5 genes in the menaquinone biosynthesis pathway are knocked out in this mutant (http://www.shigen.nig.ac.jp/ecoli/pec/quickSearch.List.DeletionsDetailAction.do?fromListFlag=true&classId=4&deId=226). E. coli strains with single gene deletions in the menaquinone biosynthesis pathway were tested for induction of growth. The results are shown in Table 1.

TABLE 1

| Deleted gene | Result |
|---|---|
| aroG | Induction of growth (multiple genes forming the product) |
| aroB | Less induction of growth |
| aroA | No induction |
| aroC | No induction |
| menF | Less induction of growth (entC can form the product) |
| menD | No induction |
| menH | Less induction of growth |
| menC | No induction |
| menE | No induction |
| menB | No induction |
| menA | Induction of growth |
| ubiE | Induction of growth |

Different quinones (Q), commercially available and/or isolated from E. coli and Micrococcus luteus were tested for induction of growth of KLE1280. The results are shown in Table 2.

TABLE 2

| Quinones | Results |
|---|---|
| Q1 (Commercially available) | No induction |
| Q2 (Commercially available) | No induction |
| Q4 (Commercially available) | No induction |
| Q9 (Commercially available) | No induction |
| Q10 (Commercially available) | No induction |
| Menaquinone4 (Commercially available) | Induction |
| Ubiquinone8 (From E. coli) | No induction |
| Ubiquinone7 (From E. coli) | No induction |
| Menaquinone8 (From E. coli) | No induction |
| Menaquinone4 (From M. luteus) | Induction |
| Menaquinone5 (From M. luteus) | Induction |
| Menaquinone6 (From M. luteus) | Induction |
| Menaquinone7 (From M. luteus) | Very less induction (late) |
| Menaquinone8 (From M. luteus) | Very less induction (late) |

The chemical structures of quinones are shown in Table 3.

TABLE 3

| Quinones | Structures |
|---|---|
| Q1 | 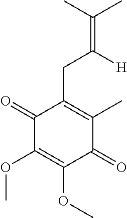 [SMILES: COC1=C(OC)C(=O)C(CC=C(C)C)=C(C)C1=O] |
| Q2 | 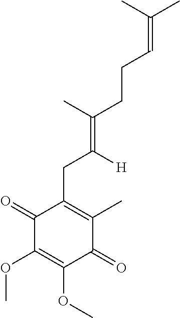 [SMILES: COC1=C(OC)C(=O)C(C/C=C(\C)CCC=C(C)C)=C(C)C1=O] |
| Q4 | 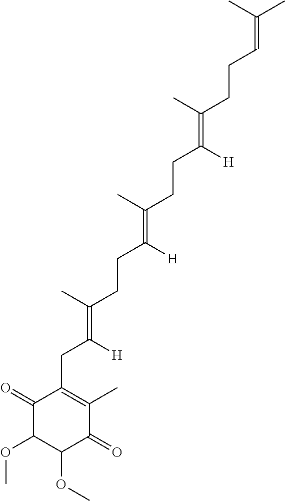 [SMILES: COC1C(=O)C(C)=C(C/C=C(\C)CC/C=C(\C)CC/C=C(\C)CCC=C(C)C)C(=O)C1OC] |
| Q9 | 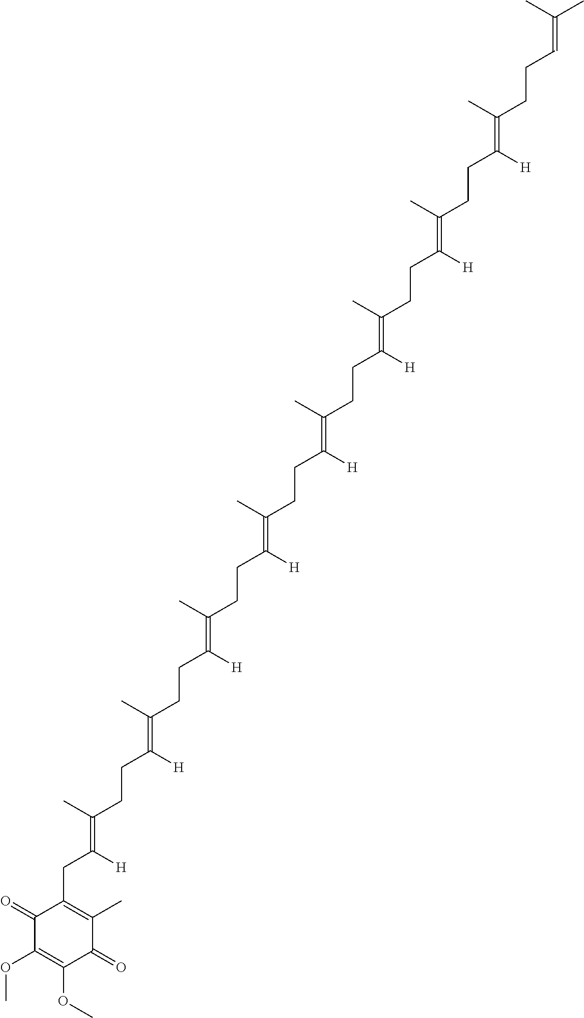 [SMILES: COC1=C(OC)C(=O)C(C/C=C(\C)CC/C=C(\C)CC/C=C(\C)CC/C=C(\C)CC/C=C(\C)CC/C=C(\C)CC/C=C(\C)CC/C=C(\C)CCC=C(C)C)=C(C)C1=O] |
| Q10 | 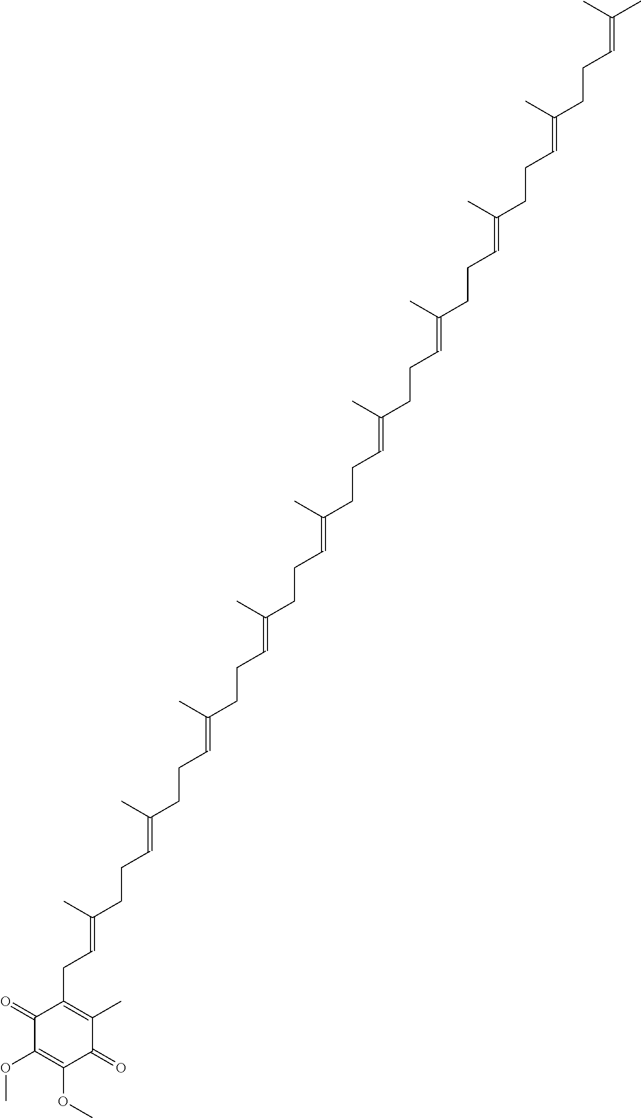 [SMILES: COC1=C(OC)C(=O)C(C/C=C(\C)CC/C=C(\C)CC/C=C(\C)CC/C=C(\C)CC/C=C(\C)CC/C=C(\C)CC/C=C(\C)CC/C=C(\C)CC/C=C(\C)CCC=C(C)C)=C(C)C1=O] |
| Menaquinone4 | 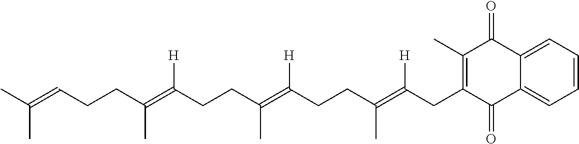 [SMILES: CC(C)=CCC/C(C)=C/CC/C(C)=C/CC/C(C)=C/CC1=C(C)C(=O)c2ccccc2C1=O] |
| Ubiquinone8 | 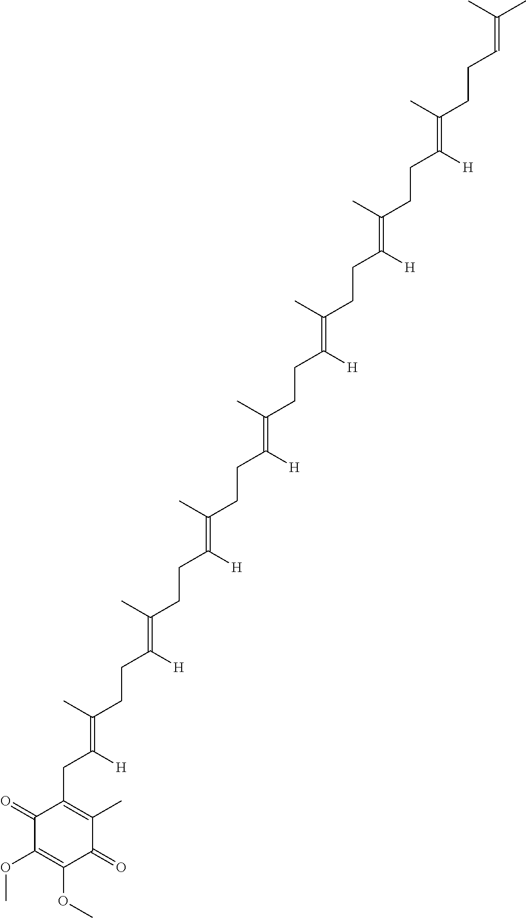 [SMILES: COC1=C(OC)C(=O)C(C/C=C(\C)CC/C=C(\C)CC/C=C(\C)CC/C=C(\C)CC/C=C(\C)CC/C=C(\C)CC/C=C(\C)CCC=C(C)C)=C(C)C1=O] |
| Ubiquinone7 | 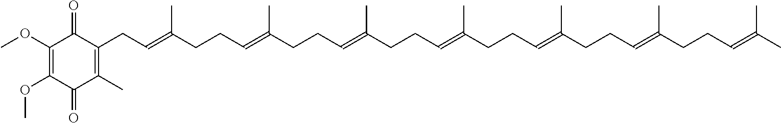 [SMILES: COC1=C(OC)C(=O)C(C/C=C(\C)CC/C=C(\C)CC/C=C(\C)CC/C=C(\C)CC/C=C(\C)CC/C=C(\C)CCC=C(C)C)=C(C)C1=O] |
| Menaquinone5 | 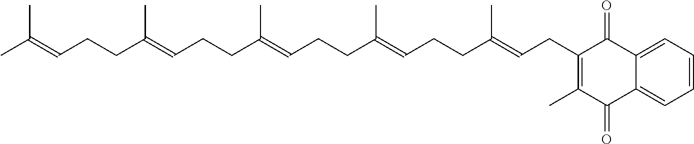 [SMILES: CC(C)=CCC/C(C)=C/CC/C(C)=C/CC/C(C)=C/CC/C(C)=C/CC1=C(C)C(=O)c2ccccc2C1=O] |
| Menaquinone6 | 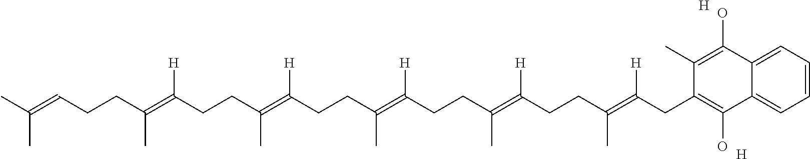 |
| Menaquinone7 | 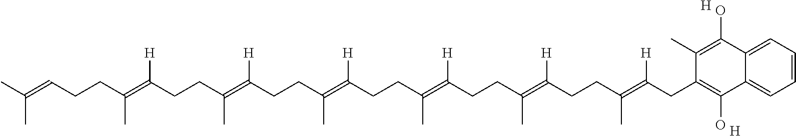 |
| Menaquinone8 | 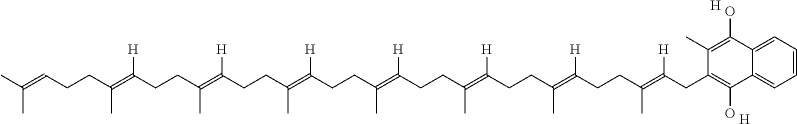 |

Menaquinone 4 (MK4) induced growth of KLE1280. One intermediate from the menaquinone biosynthesis pathway, 1,4-dihydroxy-2-naphthoate (DHNA) also showed induction of growth of KLE1280. Different concentrations of MK4 were spotted on media with and without blood (5%) or hemin (10 μg/ml) or hemoglobin (100 μg/ml). MK4 induced growth only in the presence of either of these 3 in the medium. MK4, hemin, hemoglobin or blood alone did not induce growth of KLE1280.

KLE1280 is an anaerobic organism, and quinones apparently serve as electron shuttles between components of anaerobic fermentation and external terminal acceptors. Hemin and hemoglobin apparently serve as terminal acceptors. For anaerobic uncultured microorganisms then, the growth medium should contain a quinone and a suitable electron acceptor such as hemin. The same medium can be used for anaerobic environmental microorganisms. For environmental aerobic microorganisms, a quinone may be sufficient, if its role is to complement an otherwise complete respiratory chain.

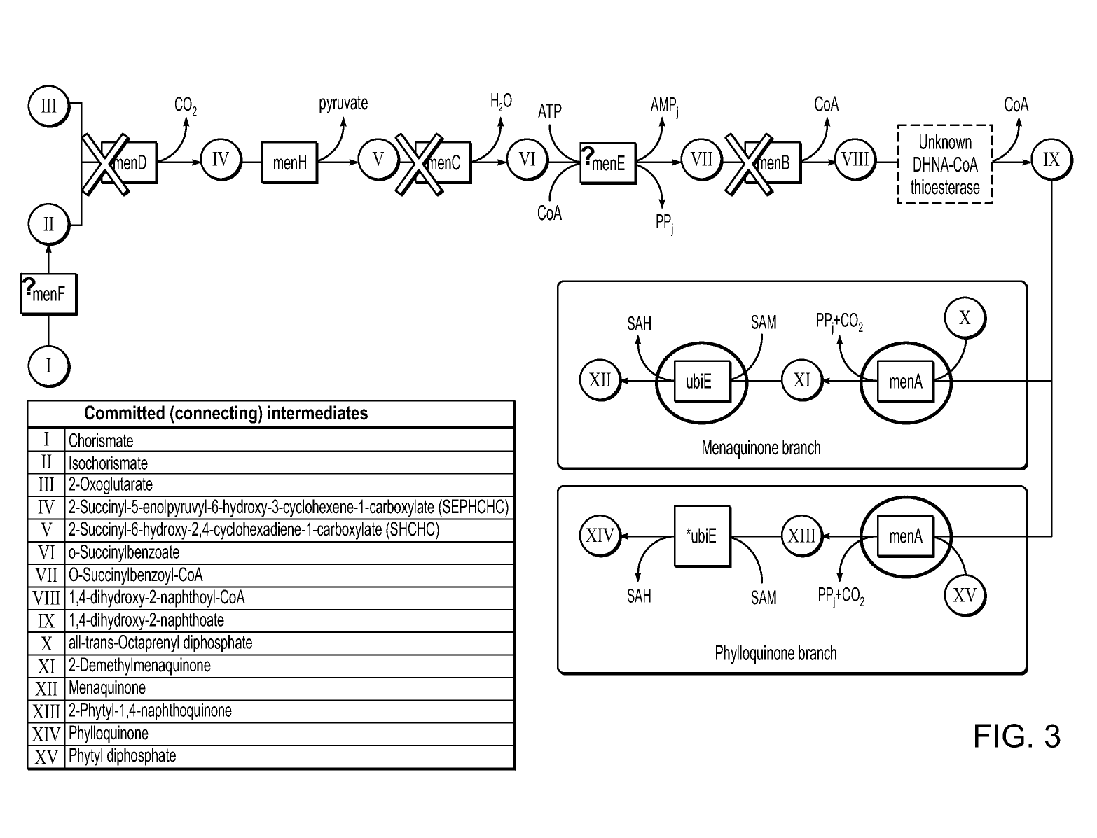

Menaquinone biosynthetic pathway of Porphyromonas gingivalis W83, from the National Microbial Pathogen Data Resource is shown in FIG. 3 (available at http://www.nmpdr.org). Genes in filled shaded boxes have been identified bioinformatically in the genome sequence of P. gingivalis W83. With the exception of menH, whose gene has not been identified in this organism, this indicates a complete pathway for menaquinone biosynthesis. This is consistent with the known ability of Porphyromonas gingivalis to produce menaquinone (Shah & Williams, 1987; herein incorporated by reference in its entirety). Superimposed on this pathway are the genes present (circled), absent (crosses), or unclear (question marks) in KLE1280. KLE1280 is missing several essential genes in this pathway, particularly menB, menC and menD. These genome sequencing results are consistent with the dependence of KLE1280 on exogenous menaquinone.

### Example 2

### Quinones Act as Growth Promoting Factors for Marine Isolates: Marine Isolate 1: Flaviramulus sp. KLE1215

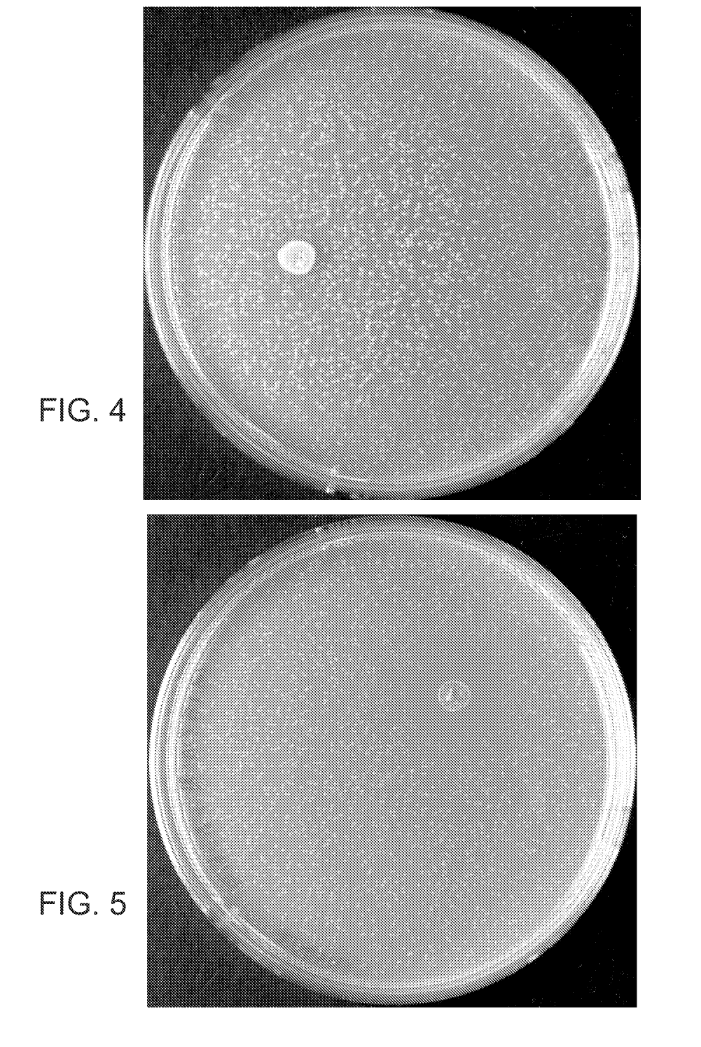

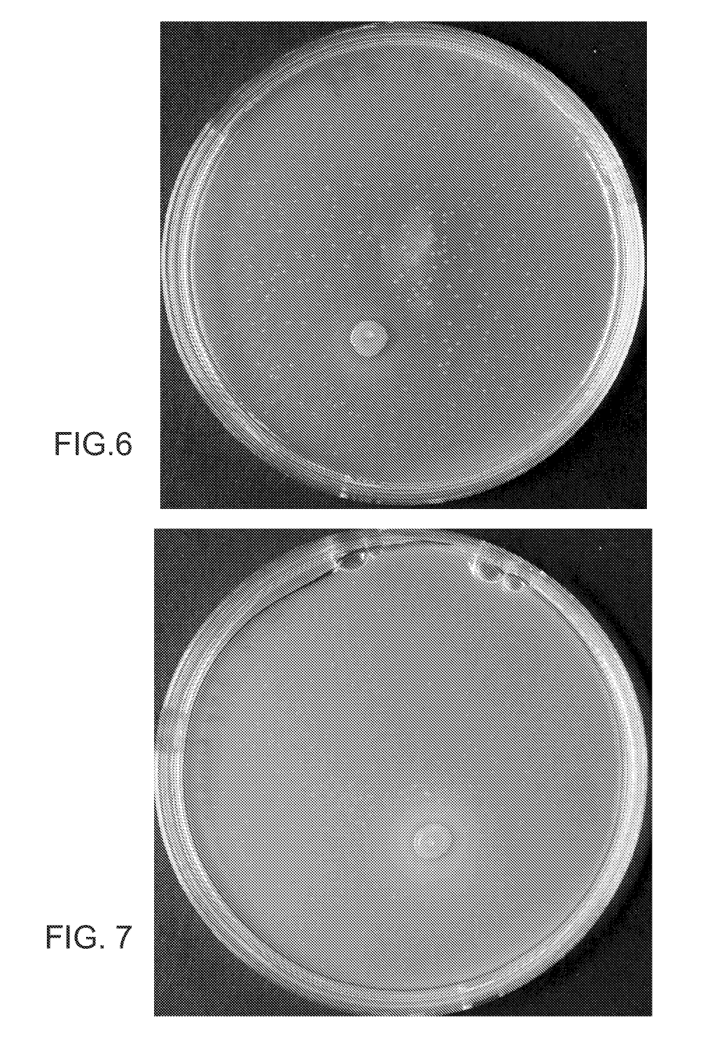

This isolate grows either poorly or not at all in the absence of exogenous quinone-like compounds. These growth promoting-compounds can be contributed by a laboratory strain of Escherichia coli or other bacteria from the environment, such as Shewanella oneidensis. As shown in FIGS. 4-7, this growth induction is eliminated when the helper strains are mutated so that they no longer produce quinones. Escherichia coli induces the growth of KLE1215. KLE1215 spread evenly on R2A with 50% artificial sea salts and 0.001% Fe(II) with a dense spot of E. coli (FIG. 4). E. coli ΔubiG, a strain deficient in quinone biosynthesis, does not induce the growth of KLE1215. KLE1215 spread evenly on R2A with 50% artificial sea salts and 0.001% Fe(II) with a dense spot of E. coli ΔubiG (FIG. 5). Shewanella oneidensis MR-1 ZK2719 induces the growth of KLE1215. KLE1215 spread evenly on R2A with 50% artificial sea salts and 0.001% Fe(II) with a dense spot of Shewanella MR-1 ZK2719 (FIG. 6). Shewanella sp. ΔmenC:kan ZK2720, deficient in quinone production, does not induce the growth of KLE1215. KLE1215 spread evenly on R2A with 50% artificial sea salts and 0.001% Fe(II) with a dense spot of Shewanella sp. ΔmenC:kan ZK2720 (FIG. 7).

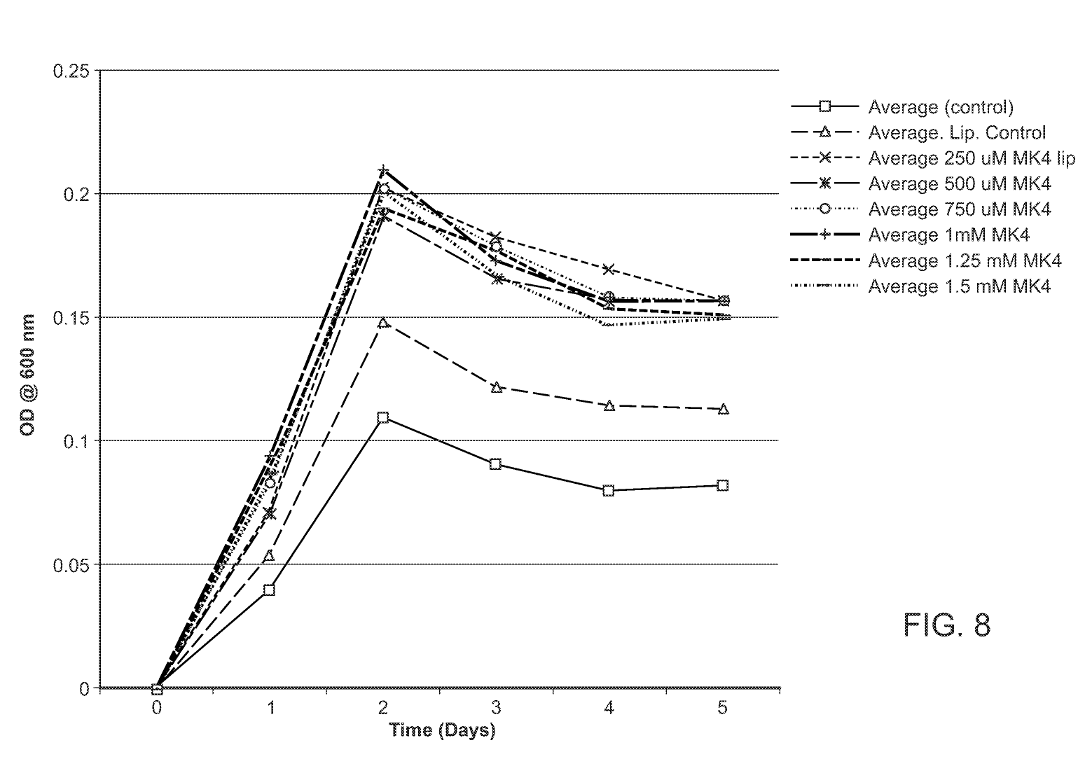

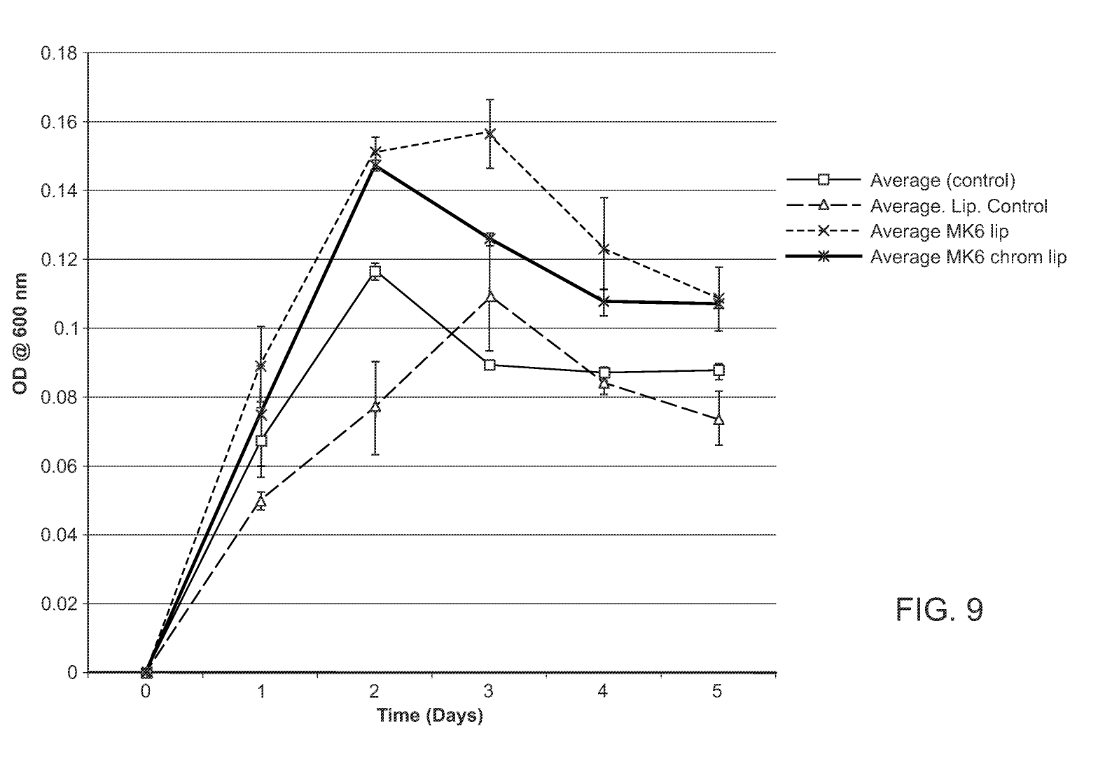

The growth of Flaviramulus sp. KLE1215 is enhanced by the addition of purified quinone-like compounds when they are delivered in liposomes. FIGS. 8 and 9 show the growth-enhancing effects of menaquinone 4 (MK4), menaquinone 6 (MK6), and menachromenal 6 (MK6 chromenols) in liposomes. Growth of KLE1215 induced with varying concentrations of MK4 delivered in liposomes. 5 ml liquid cultures of KLE1215 were set up with no supplement (squares), empty liposomes as liposome control (triangles), liposomes loaded with 250 μM MK4 (cross), liposomes loaded with 500 μM MK4 liposomes (asterisk), liposomes loaded with 750 μM MK4 (circle), liposomes loaded with 1 mM MK4 (vertical line), liposomes loaded with 1.25 mM MK4 (dark line), and liposomes loaded with 1.5 mM MK4 (light line) is shown in FIG. 8. R2A broth (without yeast extract) with 50% artificial sea salts supplemented with 0.001% Fe(II) was used for these cultures. Values represent the average of two independent cultures of KLE1215 in R2A broth with 50% artificial sea salts supplemented with 0.001% Fe(II). FIG. 9 shows growth of KLE1215 is induced by MK6 and MK6 chromenol purified from KLE1215 delivered in liposomes. 5 ml liquid cultures of KLE1215 were prepared with nothing added (squares), empty liposome as liposome control (triangles), liposomes loaded with MK6 from KLE1215 (cross), and liposomes loaded with MK6 chromenols from KLE1215 (asterisk). R2A broth (without yeast extract) with 50% artificial sea salts supplemented with 0.001% Fe(II) was used for these cultures. Values represent the average of independent cultures of KLE1215 in R2A broth with 50% artificial sea salts supplemented with 0.001% Fe(II).

### Example 3

### Quinones Act as Growth Promoting Factors for Marine Isolates: Marine Isolate 2: Bizinio sp. KLE1402

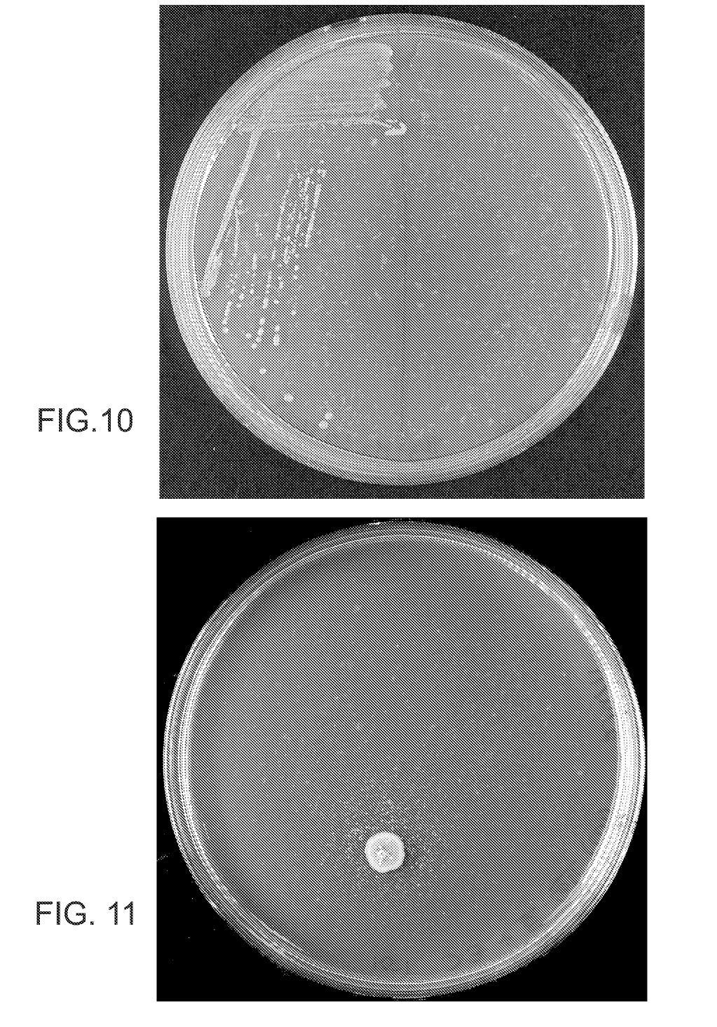

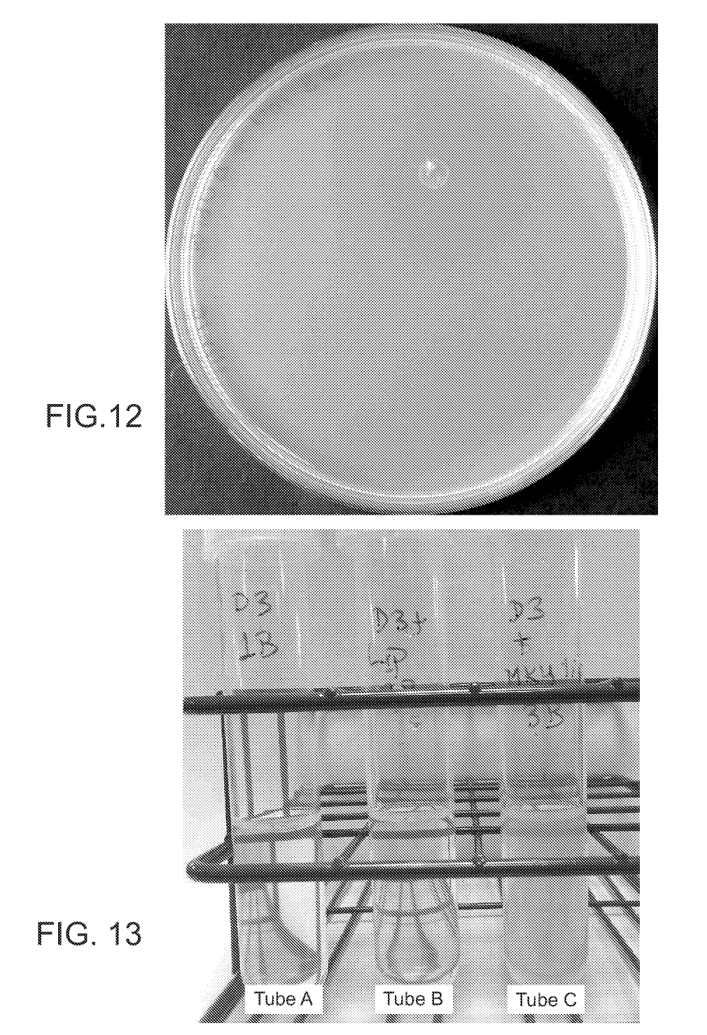

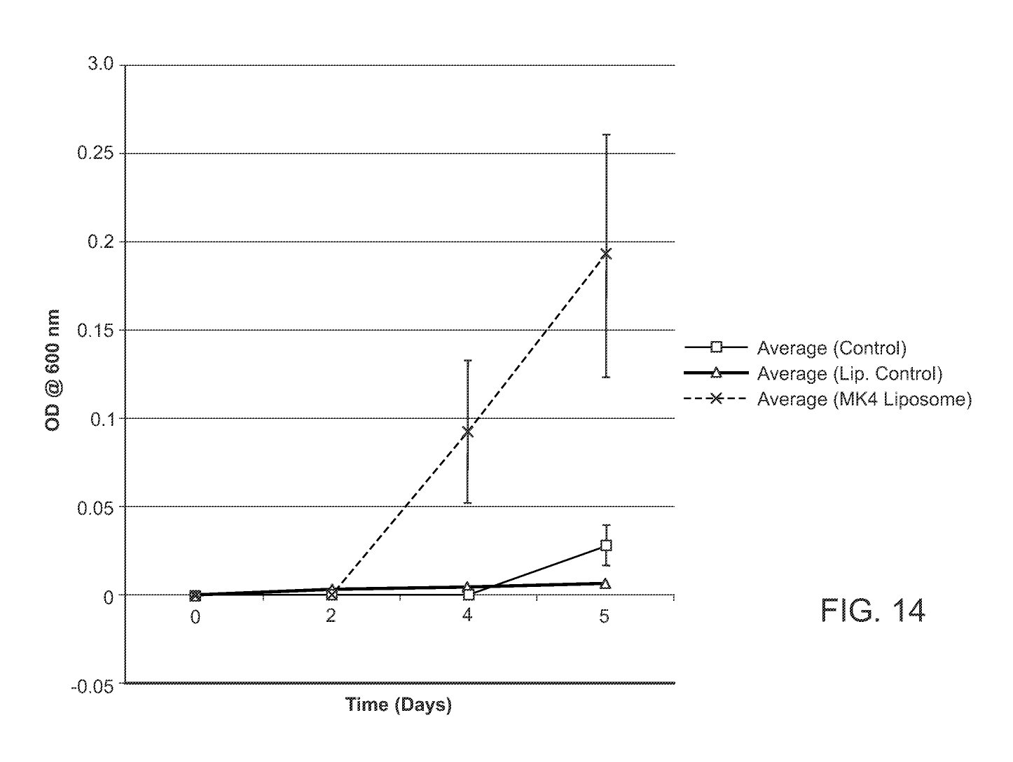

This isolate shows complete dependence on exogenous quinones for growth. FIG. 10 shows growth induction by a helper bacterium from the natural environment. FIGS. 11 and 12 show that laboratory E. coli can induce the growth of KLE1402, but that an E. coli strain unable to synthesize quinones cannot, suggesting that the growth factor is a quinone. FIGS. 13 and 14 show that purified MK4 delivered by liposomes can induce the growth of KLE1402.

An Aerobic Helper-Dependent pair isolated from marine sand biofilm is shown in FIG. 10. Two isolates were streaked for isolation; the culturable helper on the left induces the growth of unculturable KLE1402 on the right only when it is close proximity. The closest relative to the dependent KLE1402 is Bizionia echini (96.6% Identity according to 16S rRNA gene sequence), from the family Flavobacteriaceae. The closest relative to the helper KLE1403 is Ruegeria lacuscaerulensis (98.4% identity by 16S rRNA gene sequence), from the class Alphaproteobacteria. E. coli induces the growth of KLE1402 (FIG. 11). KLE1402 spread evenly on R2A with 50% artificial sea salts and 0.001% Fe(II) with a dense spot of E. coli. E. coli ΔubiG, a strain deficient in quinone biosynthesis, does not induce the growth of KLE1402 (FIG. 12). KLE1402 spread evenly on R2A with 50% artificial sea salts and 0.001% Fe(II) with a dense spot of E. coli ΔubiG. KLE1402 grows with the addition of quinone-loaded liposomes (FIG. 13). Cells of KLE1402 were used to inoculate culture tubes with 5 ml R2A broth with 50% sea salts and 0.001% Fe(II). Tube A: no supplement, Tube B: Empty liposomes added, Tube C: Liposomes loaded with 500 μM MK4 added. KLE1402 grows with the addition of quinone-loaded liposomes (FIG. 14). 5 ml liquid cultures of KLE1402 were set up with no supplement (squares), empty liposomes as liposome control (triangles), liposomes loaded with 500 μM MK4 (cross). R2A broth with 50% artificial sea salts supplemented with 0.001% Fe(II) was used as media for these cultures. Cells from glycerol stocks stored at −80° C. were resuspended in 2% artificial sea salts solution and this was used to inoculate the culture tubes. The experiment was done in triplicate. The error bars represent one standard deviation.

### Example 4

### Quinone-Like Compounds as Growth Factors for Gut Bacteria—Isolation of Gut Bacterium KLE1255

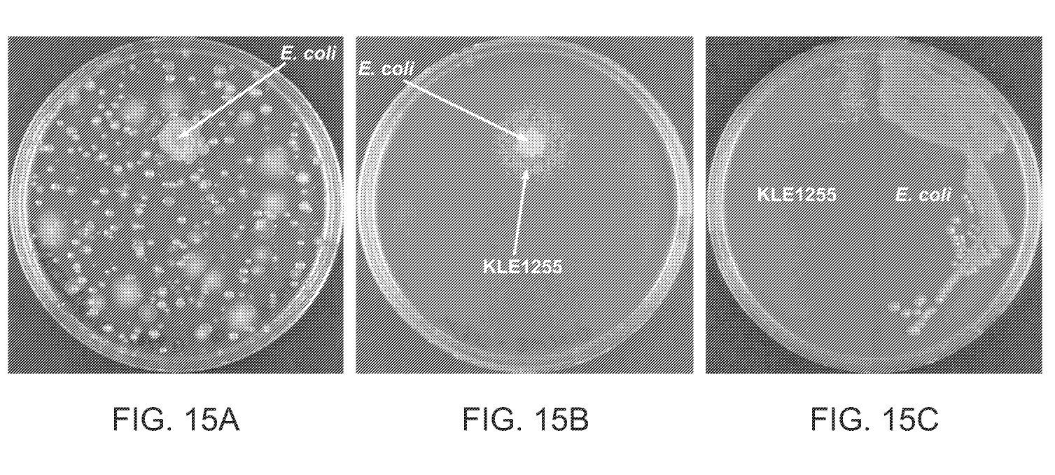

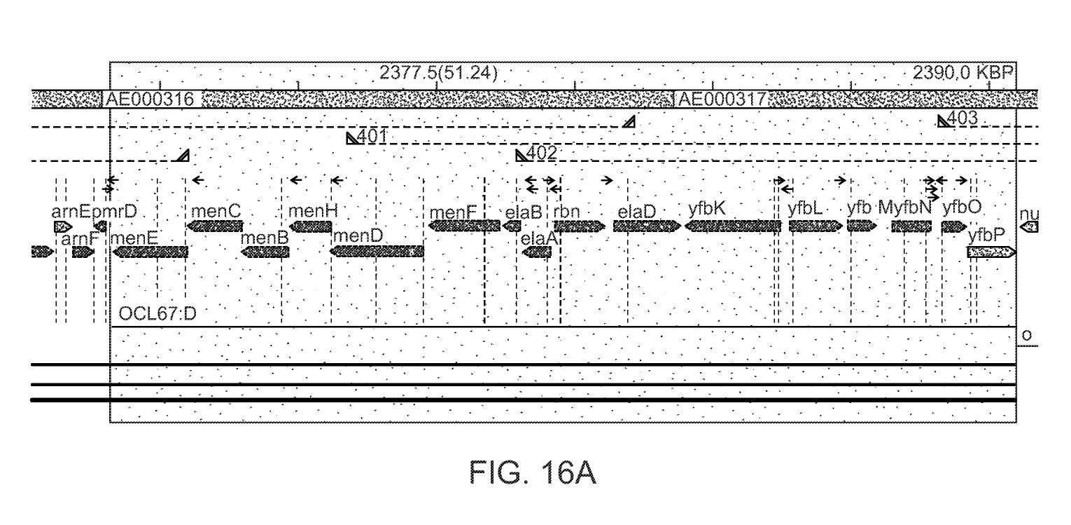

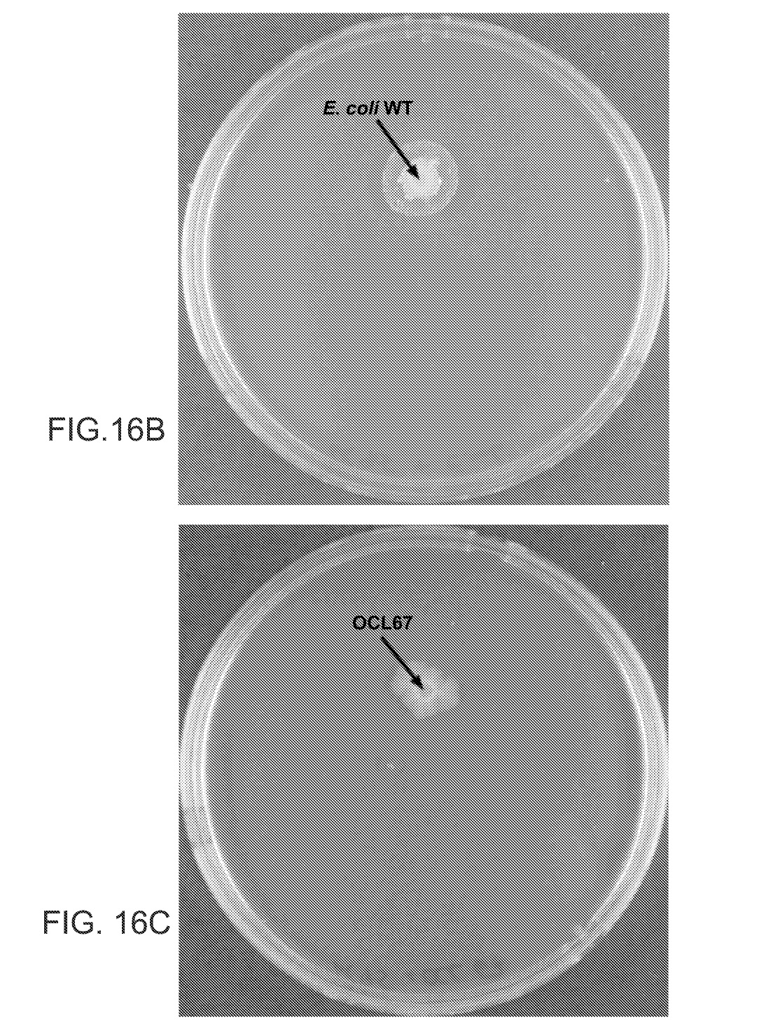

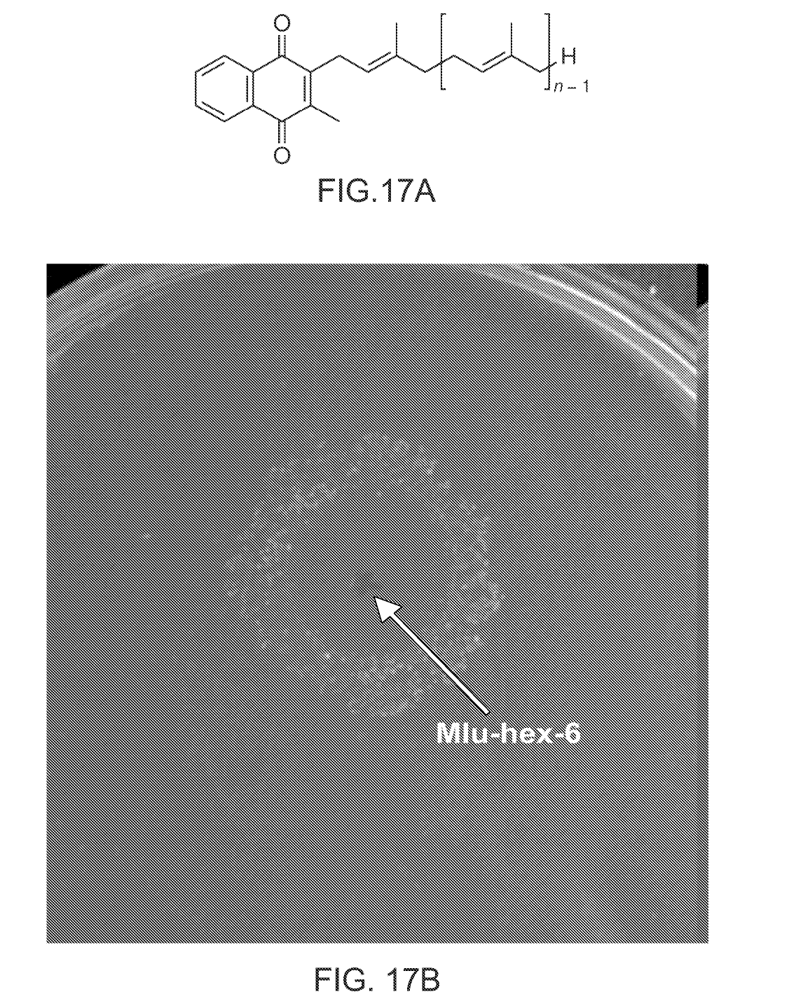

Strain KLE1255 was isolated from human feces in co-culture with Escherichia coli. KLE1255 was identified by 16S rRNA gene sequencing as a relative of Faecalibacterium prausnitzii ATCC 27768^{T}. The isolate was isolated on BHI medium supplemented with yeast extract, cysteine and hemin (BHIych) and only grew in close proximity to the E. coli helper. FIG. 15 shows the isolation and dependent growth of KLE1255. FIG. 15A shows the original isolation plate showing colonies from a fecal sample growing on BHIych with a spot of E. coli. Colonies growing in close proximity to the E. coli spot were picked, and tested for dependency on the helper, by spreading the potential dependent isolate evenly onto the plate and spotting E. coli (FIG. 15B), and by streaking the two organism close to each other (FIG. 15C). FIG. 16 shows that an E. coli mutant that does not make menaquinone does not induce the isolate. Specifically, E. coli deletion mutant OCL67 fails to induce growth of Faecalibacterium sp. KLE1255. The image shows the chromosomal region deleted in E. coli strain OCL67 (16.4 kb, shaded) (PEC database: www.shigen.nig.ac.jp/ecoli/pec/index.jsp) (FIG. 16A). The deletion includes 16 genes, six of which (menBCDEFH) make up the menaquinone biosynthesis operon. A tight ring of growth of KLE1255 can be observed around wild-type E. coli (FIG. 16B). No growth of KLE1255 can be observed with the E. coli mutant OCL67 (FIG. 16C). Single mutant strains showed that the loss of growth induction was due to the loss of the menaquinone genes. FIG. 17 shows that purified quinone-like compounds induce it to grow. Menaquinone-like compounds from Micrococcus luteus supernatant act as growth factors. M. luteus supernatant was extracted with hexane and the extract further fractionated. Four fractions that induced growth of KLE1255 were obtained. List of fractions from M. luteus supernatant hexane extract that induced the growth of KLE1255 (FIG. 17A). Two of the fractions contain menaquinone 4 and preliminary data suggest that fraction Mlu-hex-6 also contains a menaquinone-like compound. Structure of menaquinone is shown in FIG. 17B. The side chain consists of isoprenoid residues and can vary in length. n specifies the number of isoprenoid repeats. Growth induction of KLE1255 by the fraction containing a menaquinone-like compound is shown in FIG. 17C.

This invention has allowed growth of a difficult to grow bacterium and can be used to grow other uncultivated bacteria from the environment.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### Equivalents

Those skilled in the art will recognize, or be able to ascertain, using no more than routine experimentation, numerous equivalents to the specific substances and procedures described herein. Such equivalents are considered to be within the scope of this invention, and are covered by the following claims.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways to obtain additional embodiments within the scope and spirit of the invention.

1. A method for cultivating or isolating a microorganism, comprising providing at least one quinone as a growthfactor.

2. The method of claim 1, wherein the quinone is delivered in liposomes.

3. The method of claim 1, further comprising providing a helper strain microorganism.

4. The method of claim 3, wherein the quinone is produced by the helper strain microorganism.

5. The method of claim 1, wherein the the quinone is selected from the group consisting of MK4, MK5, MK6, and DHNA.

6. The method of claim 1, wherein the cultivated or isolated microorganism is selected from the group consisting of Flaviramulus sp., Bizinio sp., Porphyromonas sp., and Faecalibacterium sp.

7. The method of claim 6, wherein the cultivated or isolated microorganism is selected from the group consisting of Flaviramulus sp. KLE1215, Bizinio sp. KLE1402, Bizionia echini, Porphyromonas sp. KLE1280, Porphyromonas catoniae, Porphyromonas gingivalis, Faecalibacterium prausnitzii and Faecalibacterium sp. KLE1255.

8. The method of claim 3, wherein the helper strain microorganism is selected from the group consisting of Escherichia coli, Shewanella oneidensis, Ruegeria lacuscaerulensis, and Micrococcus luteus.

9. A method for treating a mammal having a deficiency in symbionts, the method comprising administering to the mammal a therapeutically effective amount of at least one quinone.

10. The method of claim 9, wherein quinone is delivered in liposomes.

11. The method of claim 9, further comprising administering to the mammal a helper strain microorganism.

12. The method of claim 11, wherein the quinone is produced by the helper strain microorganism.

13. The method of claim 9, wherein the quinone is selected from the group consisting of MK4, MKS, MK6, and DHNA.

14. The method of claim 9, wherein the symbionts are selected from the group consisting of Flaviramulus sp., Bizinio sp., Porphyromonas sp., and Faecalibacterium sp.

15. The method of claim 14, wherein the symbionts are selected from the group consisting of Flaviramulus sp. KLE1215, Bizinio sp. KLE1402, Bizionia echini, Porphyromonas sp. KLE1280, Porphyromonas catoniae, Porphyromonas gingivalis, Faecalibacterium prausnitzii and Faecalibacterium sp. KLE1255.

16. The method of claim 11, wherein the helper strain microorganism is selected from the group consisting of Escherichia coli, Shewanella oneidensis, Ruegeria lacuscaerulensis, and Micrococcus luteus.
